(19) United States
(12) Patent Application Publication
(10) Pub. No.: US 2010/0076366 A1
(43) Pub. Date: Mar. 25, 2010

(54) MODULAR SPINAL FLUID FLOW REGULATION DEVICE AND METHOD

(75) Inventors: Fraser Cummins Henderson, SR., Upper Marlboro, MD (US); John W. Newman, Newtown Square, PA (US)

Correspondence Address: KNOBLE, YOSHIDA & DUNLEAVY, EIGHT PENN CENTER, SUITE 1350, 1628 JOHN F KENNEDY BLVD, PHILADELPHIA, PA, 19103, US

(73) Assignee: POLARIS BIOTECHNOLOGY, INC., Newtown Square, PA (US)

(21) Appl. No.: 12/562,926

(22) Filed: Sep. 18, 2009

Related U.S. Application Data
(60) Provisional application No. 61/098,671, filed on Sep. 19, 2008.

(51) Int. Cl. A61M 1/00

(52) U.S. Cl. 604/9

(57) ABSTRACT

A shunt system for telemetrically measuring, regulating and/or adjusting cerebrospinal fluid flow rate, intercranial pressure, intraspinal pressure and/or intraventricular pressure and a method for use. The shunt system includes a shunt assembly, a first catheter and a second catheter that may be implanted using a novel introducer assembly. In addition to regulating fluid pressure and flow rate, the shunt system may also be used to deliver therapeutic compositions.

### CROSS-REFERENCE TO RELATED APPLICATIONS

This application is a non-provisional application of and claims benefit of priority to U.S. Provisional Patent Application No. 61/098,671, filed Sep. 19, 2008, the entire disclosure of which is incorporated herein by reference.

### BACKGROUND OF THE INVENTION

1. Field of the Invention

The present invention is directed to a surgically implantable shunt system and methods for use thereof. Specifically, the invention relates to a shunt system that is capable of telemetric pressure and/or flow rate detection and regulation.

2. Description of the Related Technology

In a healthy individual, cerebrospinal fluid (CSF) is continuously produced by the choroid plexus within the ventricles. Generally, about 100 cc to about 600 cc of CSF is secreted in the brain each day, the amount of which may be affected by numerous factors, such as endocrine changes, water volume in the body, intervascular pressure, proteins in the blood, infections and antibiotics. Circulates through the ventricles and around the brain and spinal cord, the CSF eventually drains into the circulatory system thereby maintaining intercranial pressure (ICP) and intraspinal pressure (ISP). Specifically, the CSF flows from the lateral ventricles via the foramina of Monro into the third ventricle, and then the fourth ventricle via the cerebral aqueduct in the brainstem. From there it normally passes into the central canal of the spinal cord or into the cisterns of the subarachnoid space via three small foramina, the central foramen of Magendie and the two lateral foramina of Luschka.

Anatomical deformities and conditions that block or restrict CSF flow through the ventricles or subarachnoid space can disrupt normal CSF flow. When the CSF flow is impeded, the continued production of CSF will cause an increase in ICP and ISP as the fluid collects within the ventricles or subarachnoid space, thereby causing various problematic physiological conditions.

Currently, there is a large underserved population of patients who have an underlying problem of elevated ICP, including patients diagnosed with benign intracranial hypertension, normal pressure hydrocephalus, tarlov cyst syndrome, chiari malformation, chronic pseudomeningocoele or communicating hydrocephalus. Elevated ICP has also been noted in a subset of patients diagnosed with alzheimer's disease, chronic fatigue syndrome, myofascial syndrome, fibromyalgia, and tethered cord syndrome. With the exception of communicating hydrocephalus, a definitive diagnosis for elevated ICP remains problematic; consequently, this condition is not effectively treated.

Additionally, current treatments for irregular ICP and ISP, which is limited to implanting ventriculo-peritoneal shunts (VP shunt) and lumbo-peritoneal shunts (LP shunt), produce poor results and have high failure rates. In general, conventional VP and LP shunts fail shortly after implantation for a variety of reasons, including catheters pulling out of the spinal fluid space or the abdominal space, infection, or need for revision due to over drainage or under-drainage.

Conventional VP shunt, which requires creating a cranial burr hole, can cause brain injuries, scalp injuries, infection, undesirable cosmetic consequences and shunt failure. VP shunts also undesirably restrict the patient's activity level and lifestyle.

Conventional LP shunts are plagued with high failure rates requiring surgical correction or replacement. For example, LP shunts commonly cause spinal fluid headaches due to poor maintenance of pressure in the lumbar subarachnoid space, and consequently, require surgical revision. Additionally, complications may arise during implantation causing injuries, such as nerve injuries, bowel perforations, ligament injuries or abdominal muscle injuries, and infection. Caused in part by usage of radiolucent catheters and tubes that cannot be readily observed via x-ray unless a barium impregnated elastomer is used in the catheter construction, these injuries may arise as a result of inadvertent catheter placement. For example, a catheter intended to pass into a spinal fluid space, such as the subarachnoid space, may inadvertently pass into the epidural space or may project into normal or scarred nerve roots, causing nerve damage. Furthermore, many conventional LP shunts are orientation specific; improper positioning of these shunts may result in over drainage or under-drainage. Conventional shunts also have undesirable excessive profiles requiring long incisions for implantation as well as causing unsightly scarring, subcutaneous protrusions, and pain. Additionally, sutures are typically required to secure placement of the LP shunt; these sutures may damage or penetrate the shunt or catheters, requiring replacement of the entire LP shunt system.

Furthermore, existing LP shunts provide no reliable quantitative means to measure or monitor CSF flow rate or pressure without performing a lumbar puncture. These shunts also fail to provide a mechanism for quantitatively adjusting or setting CSF flow rate or pressure. At most, conventional shunts, such as the Integra Spetzler shunt, achieves a degree of pressure regulation through the use of small diameter tubing with a high resistance and separate miter valve for additional hydraulic resistance to achieve a low, medium, or high pressure. Although there currently exist shunts, such as those incorporating the Medtronic Delta and the Strata Valves, that are capable of maintaining a constant intraventricular pressure (IVP) within a normal range of physiologic pressure, these shunts cannot be adjusted based on an actual measured ISP or ICP and patient symptoms. Therefore, excessive fluid may be inadvertently removed, which in addition to causing severe disabling headaches, may alter normal cerebrospinal fluid physiology.

Current lumbar shunts are also poorly adapted for infusing drugs into the spinal fluid, which may be useful in treating conditions such as carcinomatous meningitis, multiple sclerosis and hormone deficiencies.

In view of the aforementioned deficiencies of traditional shunts, there is a need to develop an improved shunt system that is capable of regulating and restoring normal CSF flow as well as normal ICP, ISP and IVP.

### SUMMARY OF THE INVENTION

The invention includes a novel shunt system and introducer system. In a first aspect, the shunt system includes: a first catheter having a first tube body, a sensor positioned on the first tube body, and a shunt assembly configured to be surgically implanted in a bodily cavity and removably attached to the first catheter, wherein the shunt assembly includes a reservoir, a valve assembly that controls fluid flow through the shunt assembly, and a first controller operatively associated with the valve assembly and sensor; and a second catheter removably attached to the shunt assembly comprising a second tube body.

In a second aspect, the invention is directed to a shunt system including: a shunt assembly configured to be surgically implanted in a bodily cavity, wherein the shunt assembly includes a reservoir, a valve assembly that controls fluid flow through the shunt assembly, wherein said valve assembly comprises a pump capable of forcing fluid out of the shunt assembly, a first controller operatively associated with the valve assembly, a sensor capable of measuring pressure or flow rate, wherein the sensor is operatively associated with the controller, a first signal conditioner operatively associated with the controller, and a first transponder operatively associated with the first signal conditioner and capable of receiving a signal; and a reader assembly positioned outside the bodily cavity, wherein the reader assembly includes a second signal conditioner, a second transponder unit operatively associated with the second signal conditioner and capable of transmitting the signal, and a second controller operatively associated with the second signal conditioner.

In a third aspect, the invention is directed to an introducer system including: an introducer adapted to introduce a medical device into a body, wherein the introducer has a handle, a sleeve attached to a distal end of the handle, wherein the sleeve includes an introducer channel for receiving the medical instrument, and a recess positioned on an exterior surface of the sleeve, wherein the recess is substantially parallel to the introducer channel and configured to guide surgical incisions.

### BRIEF DESCRIPTION OF THE DRAWINGS

FIG. 1 is a schematic diagram of an exemplary lumbarperitoneal shunt system of the present invention.

FIG. 2(a) is a perspective view of an exemplary intraspinal catheter of the present invention.

FIG. 2(b) is a cross section of the intraspinal catheter of FIG. 2(a) at line A-A, showing the conductive wires embedded within the tubular body of intraspinal catheter.

FIG. 3(a) is a cross-section of an exemplary telemetric shunt assembly, showing a micropump valve assembly.

FIG. 3(b) is a cross-section of another exemplary telemetric shunt assembly, showing a control circuit housed in a separate chamber within a reservoir of the shunt assembly.

FIG. 3(c) is a schematic diagram of an exemplary reader showing a reader positioned outside a patient's body that is capable of telemetrically communicating with and recharging a battery of an exemplary shunt assembly that is positioned within a bodily cavity.

FIG. 3(d) is another schematic diagram of an exemplary reader showing a reader positioned outside a patient's body that is capable of telemetrically communicating with and charging a shunt controller to run a piezoelectric micro pump and a micro valve.

FIG. 4 is a perspective view of an exemplary introducer assembly, showing an introducer removably holding a bore needle and stylet.

FIG. 5(a) is a schematic diagram of the lower back and lumbar spine of a patient, showing the incision points where the introducer assembly of FIG. 4 is used to insert an intraspinal catheter.

FIG. 5(b) is a schematic diagram showing the insertion of a bore needle and stylet into the lumbar spine using the introducer assembly of FIG. 4.

FIG. 5(c) is a close-up view of FIG. 5(b), showing insertion of a needle, stylet tip and electrical potential sensor in a subarachnoid space.

FIG. 5(d) is a close-up view of the sleeve, recess and central channel of an exemplary introducer holding an intraspinal catheter.

FIG. 5(e) is a close-up view of the schematic diagram of FIG. 5(a) showing a scalpel cutting along the recess of an introducer assembly.

### DETAILED DESCRIPTION OF THE PREFERRED EMBODIMENT(S)

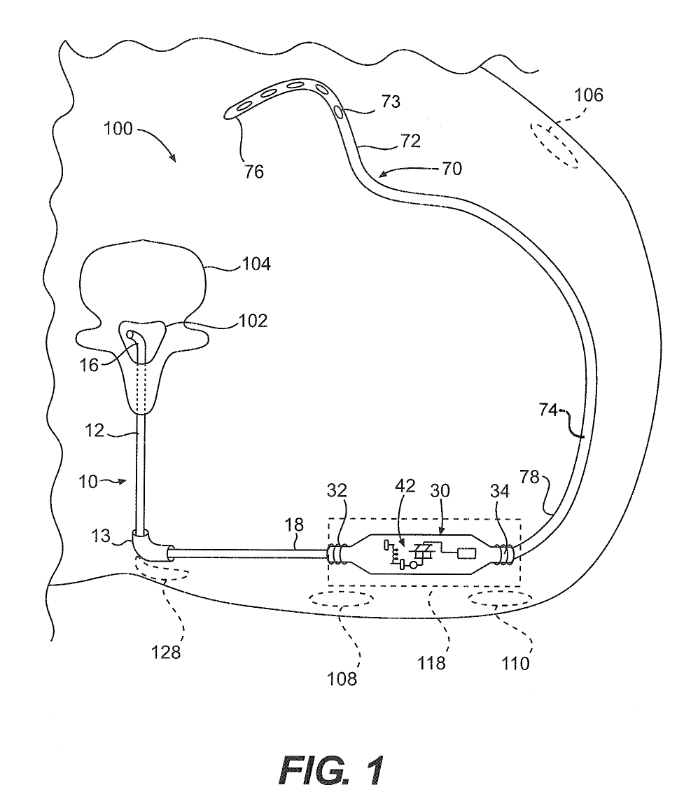

Referring now to the drawings, wherein like reference numerals designate corresponding structure throughout the views, and referring in particular to FIG. 1, shunt system 100 of the present invention includes intraspinal catheter 10, shunt assembly 30, and a drainage catheter 70. Shunt system 100 of the present invention is capable of telemetrically measuring, regulating, setting and/or otherwise adjusting cerebrospinal fluid (CSF) flow rate and/or pressure in real time. At any point in time or over an extended period of time, shunt system 100 may be used to measure and monitor CSF flow rate and/or fluid pressure, such as intercranial pressure (ICP), intraspinal pressure (ISP) and/or intraventricular pressure (IVP), at any point along intraspinal catheter 10. Based on this information, shunt system 100 can be remotely instructed to increase fluid drainage in order to lower CSF pressure or alternatively instructed to decrease drainage in order to avoid abnormally low ICP or ISP, which can cause subarachnoid hemorrhages or parenchyma collapse. Shunt system 100 can also be used as a drug delivery apparatus by blocking CSF passage to allow for infusion of one or more therapeutic compositions into shunt assembly 30. In an exemplary embodiment, shunt system 100 of the present invention may be configured as a fully modular system that is MRI compatible/visible, highly durable, easily secured to surrounding bodily tissue, resistant to infection, and positionable in any region of the body without any orientational restrictions.

Shunt system 100 includes an intraspinal catheter 10 having any shape or dimension. Additionally, intraspinal catheter 10 may be fabricated from any biocompatible material suitable for being implanted in and attached to a region of the spinal canal, such as the subarachnoid. In an alternative embodiment, intraspinal catheter 10 may be positioned in any region of the body, including the ventricles. In an exemplary embodiment, intraspinal catheter 10 may have a length of about 25 cm or more and may be about 1 mm to about 3 mm in diameter to facilitate passage through the spinal canal. Intraspinal catheter 10 may also be fabricated from a radiopaque, non-ferrous material that is substantially flexible or slightly stiff to facilitate passage into the spinal canal.

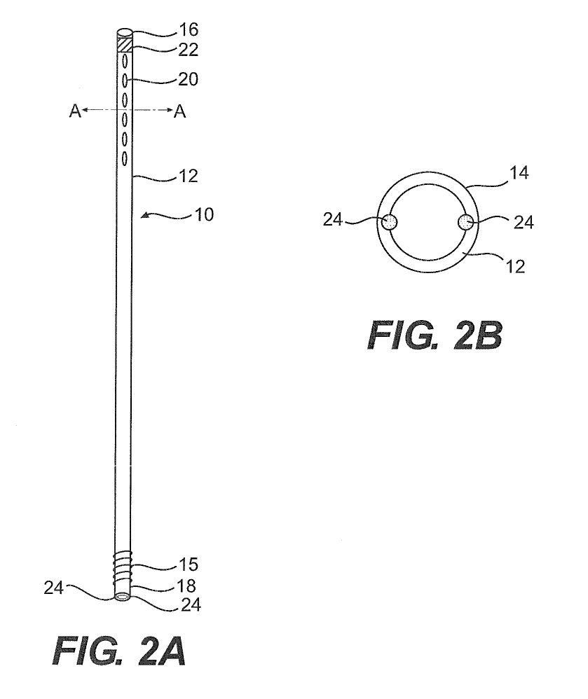

As shown in the exemplary embodiment of FIGS. 2(a)-2(b), intraspinal catheter 10 has a tubular body 12 having an outer surface 14, a distal end 16, proximal end 18 and one or more apertures 20 positioned therein to permit ingress of fluid into intraspinal catheter 10. In one embodiment, at least the segment of outer surface 14 to be inserted within the spinal canal, preferably, about 10 cm or less, more preferably about 7 cm or less of tubular body 12 adjacent to and including distal end 16, may be substantially smooth to facilitate insertion within the spinal canal. One or more regions of outer surface 14 to be positioned outside the spinal canal may be textured to prevent coiling or sliding of tubular body 12 relative to adjacent tissue. In an exemplary embodiment, about 1 cm to about 20 cm, preferably, about 1 cm to about 18 cm, more preferably, about 1 to about 15 cm, and most preferably, about 1 cm to about 10 cm of tubular body 12 adjacent to and including proximal end 18 may be textured. Exemplary textured surfaces 15 include protuberances, such as ribs, nubs, teeth, or barbs, any other roughness means or combinations thereof. In an exemplary embodiment, one or more textured surfaces 15 may have a coefficient of friction of about 0.2 to about 1.0, preferably, about 0.6 to about 1.0, under dry friction or lubricated static friction conditions between textured surface 15 and the surrounding subcutaneous tissue.

Additionally, as shown in FIG. 1, tubular body 12 may include one or more elbows 13 having a prefabricated angle. In an exemplary embodiment, the angle conforms to the anatomical site of insertion within a patient, preferably between 45 degrees to about 120 degrees. Elbow 13 may be constructed from the same or different material than tubular body 12. In an exemplary embodiment, elbow 13 is constructed from a reinforced structure having a greater stiffness than the adjacent regions of tubular body 12 and fabricated from a biocompatible, radiopaque material. Moreover, one or more regions of outer surface 14 along elbow 13 may include textured surfaces 15 to facilitate attachment to surrounding tissue.

Tubular body 12 also includes one or more sensors 22 suitable for detecting fluid pressure and/or fluid flow rate of the environment adjacent to sensor 22, such as the pressure and/or spinal fluid flow rate within the lumbar subarachnoid space. In an exemplary embodiment, the sensor 22 may have tapered edges, rounded corners, no sharp points or edges, and a minimal thickness of about 2 mm to about 12 mm. The sensor 22 may also be round, oval with a flattened body, disc shaped, rectangular with rounded edges, or cylindrical. Sensor 22 may be positioned at any point along tubular body 12, preferably any where on outer surface 14. In an exemplary embodiment, sensor 22 may be positioned at the tip of distal end 16, adjacent to distal end 16, or adjacent to one or more apertures 20. One or more wires 24 may conduct electrical signals encoding fluid pressure and/or flow rate measurements from sensor 22 to a control circuit 42, which, based on the detected pressure and/or flow rate, may open or close a valve of valve assembly 50 to regulate fluid pressure and/or fluid flow. As shown in FIG. 2(b), two wires 24 may be attached to, positioned along or embedded within tubular body 12 extending throughout a substantial length of intraspinal catheter 10 connecting sensor 22 to control circuit 42, which is positioned within a shunt assembly 30.

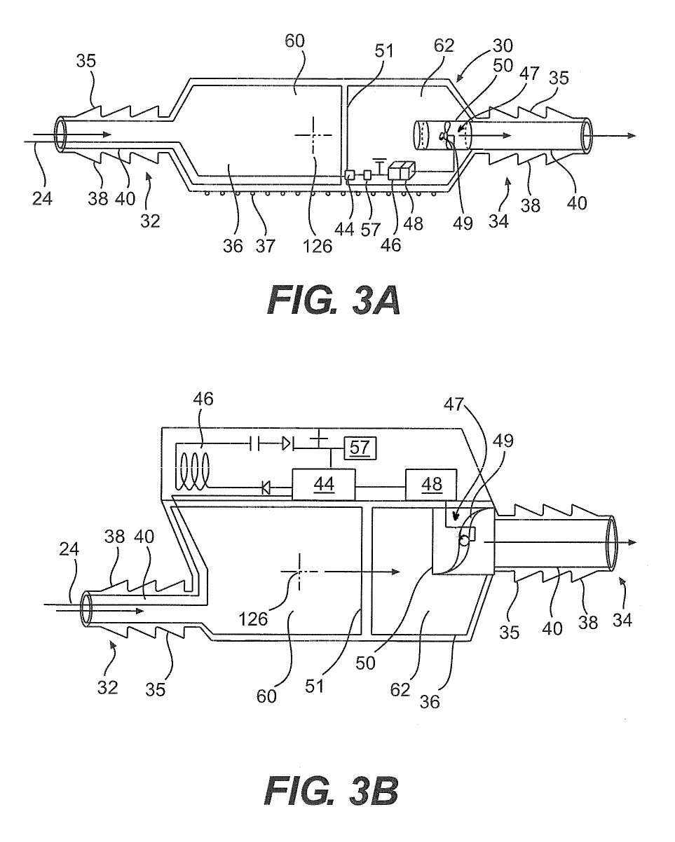

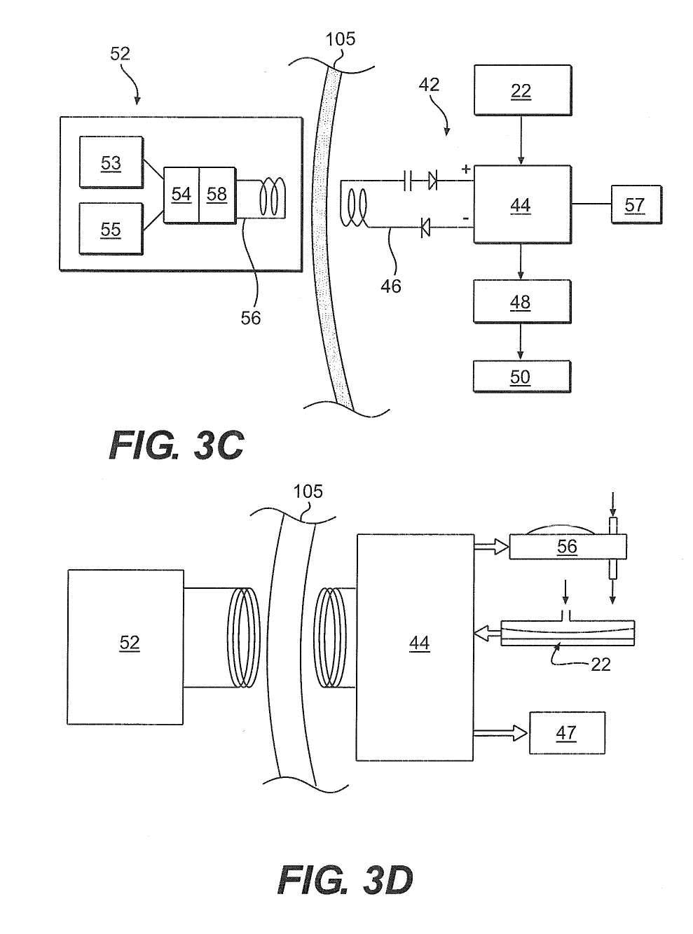

Shunt assembly 30 may be surgically implanted in any region of the body and may be positioned in any direction or orientation. FIG. 3(a) shows an exemplary shunt assembly 30 having a shunt inlet 32, shunt outlet 34, and reservoir 36. Shunt inlet 32 and outlet 34 may have the same or different dimensions and may be sized to permit entrance of a small gauge needle for the delivery of solid or liquid therapeutic compositions, sampling the CSF or assessing CSF pressure. Additionally, shunt inlet 32 and shunt outlet 34 may also be reinforced or constructed from a substantially stiffer material than adjacent reservoir 36, preferably a biocompatible radiopaque material. As shown in FIG. 3(a), shunt inlet 32 and outlet 34 may be removably and securely fastened to proximal end 18 of intraspinal catheter 10 and proximal end 78 of drainage catheter 70, respectively, without requiring a suture tie. An exterior surface 38 or interior surface 40 of shunt inlet 32 and shunt outlet 34 may include a plurality of fastening structures 35 to enhance attachment. Exemplary fastening structures 35 include notches; protuberances, such as teeth, barbs, hooks or other friction fitting means; or combinations thereof. In an exemplary embodiment, proximal ends 18, 78 of intraspinal catheter 10 and drainage catheter 70 may have a textured interior or exterior surface having mating structures, such as notches; protuberances, such as teeth, barbs or hooks; or combinations thereof, corresponding to fastening structures 35 of shunt inlet 32 and outlet 34. Proximal ends 18, 78 may also have a larger or smaller diameter than adjacent regions of tubular body 12, 72 to better fit over or within shunt inlet 32 and outlet 34. Optionally, proximal end 18 and proximal end 78 may further include tangs to facilitate attachment.

Reservoir 36 of shunt assembly 30 may have any shape, dimension or configuration and may be constructed from any suitable biocompatible material Sufficiently sized to regulate fluid flow and house the components of shunt assembly 30, reservoir 36 may have a substantially low profile, thereby minimizing deformity of the overlaying tissue and reducing patient discomfort. In an exemplary embodiment, reservoir 36 measures about 4 cm to about 10 cm in length, and the length of reservoir 36, shunt inlet 32 and shunt outlet 34 may be about 2 cm to about 10 cm. Additionally, reservoir 36 may have an exemplary volume of about 0.1 cc to about 2 cc, preferably, about 0.2 cc to about 0.4 cc, an exemplary width of about 0.5 to about 2 cm, an exemplary thickness of about 0.2 to about 1 cm, and an angular rise relative to shunt inlet 32 and/or shunt outlet 34 of about 3 degrees to about 80 degrees, preferably about 45 degrees. Furthermore, reservoir 36 may be constructed from any suitable biocompatible material, preferably, a non-ferrous radiopaque material, such as a plastic or polymer material having barium strips embedded therein. One or more regions of an exterior surface of reservoir 36 may also be textured to facilitate attachment to surrounding tissue, obviating the need for anchoring shunt assembly 30 to adjacent tissue using sutures. Exemplary textured surfaces 37 include protuberances, such as ribs, nubs, teeth, or barbs, any other roughness means or combinations thereof. In an exemplary embodiment, one or more textured surfaces 37 may have a coefficient of friction of about 0.2 to about 1.0, preferably, about 0.6 to about 1.0.

Positioned within reservoir 36 is a control circuit 42 including a shunt controller 44, a shunt transponder 46, and shunt signal conditioner 48 that is operatively associated with valve assembly 50 to control fluid flow through shunt assembly 30. As shown in the exemplary embodiment of FIG. 3(b), control circuit 42 may be positioned in a sealed chamber of reservoir 36, separate from shunt inlet chamber 60 and shunt outlet chamber 62. Alternatively, control circuit 42 may be housed within valve assembly 50.

Shunt controller 44 may be any microcontroller, microprocessor or other electronic processing unit, the primary function of which is to control the opening and closing of one or more valves 47 of valve assembly 50, thereby regulating CSF flow rate through shunt assembly 30 and CSF pressure, such as ICP, ISP and/or IVP. In an exemplary embodiment, shunt controller 44 may further control the degree to which one or more valves 47 of valve assembly 50 is opened or closed.

Shunt controller 44 regulates the opening and closing of one or more valves 47 of valve assembly 50 based on the information received from sensor 22 and/or instructions remotely transmitted to shunt transponder 46. Signals encoding the fluid pressure and/or flow rate measured by sensor 22 are conducted through wires 24 to shunt controller 44. In an exemplary embodiment, shunt controller 44 may include a pump speed log and/or log of when and to what degree one or more valves 47 are open. Shunt controller 44 analyzes this information by comparing the measured fluid pressure and/or flow rate with a preprogrammed desired fluid pressure and/or flow rate and automatically adjusts one or more valves 47 of valve assembly 50 to change the rate of fluid flow through shunt assembly 30 so as to achieve the desired preprogrammed fluid pressure and/or flow rate. In an exemplary embodiment, shunt assembly 30 may be programmed to achieve a fluid pressure of about 10 cm H₂O to about 500 cm H₂O or about 0.38 psi to about 1.15 psi. In another exemplary embodiment, shunt assembly 30 may be programmed to achieve a fluid flow rate of about 0.5 cc per hour to about 10 cc per hour.

As shown in FIGS. 3(c)-3(d), the signals encoding the fluid pressure and/or flow rate measured by sensor 22 may also be telemetrically transmitted through a patient's skin 105 to a reader 52 positioned outside the patient's body using shunt transponder 46 and shunt signal conditioner 48. Shunt transponder 46 may be any transmitter and receiver unit, such as a radio frequency transmitter/receiver or a magnetic induction coil, and shunt signal conditioner 48 may be any signal processing device or circuit that enables signal conversion, such as between electrical signals and radio frequency or magnetic inductance, signal amplification, signal encryption, signal filtering, signal isolation and/or any other signal processing means. In the exemplary embodiment of FIG. 3(c), shunt transponder 46 and a corresponding reader transponder 56 are configured as magnetic induction coils. Each transponder may include two or more magnetic induction coils oriented in different directions relative to one another to enhance signal strength and reception, as shown in U.S. Pat. No. 6,975,198, herein incorporated by reference in its entirety. Operatively associated with shunt controller 44, shunt signal conditioner 48 may be used to convert the electric signal encoding the fluid pressure and/or flow rate measured by sensor 22 to magnetic inductance. Shunt transponder 46 subsequently transmits the magnetic inductance to reader transponder 56, which is processed by reader signal conditioner 58 and reader controller 54. The information may then be relayed to a reader display unit 53 of reader 52 to be viewed by a physician. Reader controller 54 may perform calculations and data mining functions, such as compiling a history of fluid pressure and determining an average or baseline fluid pressure and/or flow rate, and subsequently display the calculations via reader display unit 53. In an exemplary embodiment, reader controller 54 may monitor fluid pressure and/or flow rate at regular intervals for several days to determine a pressure and/or flow rate baseline. Alternatively, fluid flow can be measured in the lateral position to determine baseline flow rates.

Using a user interface 55 of reader 52, a physician may also adjust or set shunt assembly 30 to achieve a desired flow rate and/or fluid pressure for a select period of time. After inputting a desired flow rate and/or fluid pressure and an applied duration, signal conditioner 58 converts the electrical signal encoding the physician's instructions into magnetic inductance, which is then transmitted by reader transponder 56 to shunt transponder 46. The signal is processed by shunt signal conditioner 48 and directed to controller 44. Controller 44 compares the instructions to the measured fluid pressure and/or flow rate of sensor 22 and adjusts one or more valves 47 of valve assembly 50 to achieve the desired fluid pressure and/or flow rate through shunt assembly 30.

A power source 57, such as a capacitor or battery, preferably a rechargeable battery, operatively associated with control circuit 42 provides power to operate the components of control circuit 42. In an exemplary embodiment, power source 57 may be either connected to or incorporated in shunt controller 44. In another exemplary embodiment, the magnetic induction coils of reader transponder 56 may be used to extracorporeally charge power source 57. Controller 44 may function as a battery charger, relay the charge received from reader 52 to battery 57. Reader 52 therefore may alternately or simultaneously communicate information with shunt assembly 30 and charge power source 57. Reader controller 54 and reader display unit 53 may therefore remotely monitor the energy level of power source 57 in addition to monitoring fluid pressure and flow rate. An exemplary embodiment of a dual magnetic induction coil transponder and battery charger is shown in U.S. Pat. No. 6,975,198, herein incorporated by reference in its entirety.

Valve assembly 50 may include one or more valves 47 operatively associated with and controlled by controller 44 to quantitatively regulate fluid flow. Exemplary valves 47 include electrically powered check valves or diaphragm valves. Valve assembly 50 can be programmed to open and close one or more valves 47 on command over one or more designated periods of time to achieve a desired CSF flow rate or fluid pressure, such as ICP, ISP and/or IVP. In an exemplary embodiment, valve assembly 50 may be programmed to open one or more valves 47 at a specified pressure in the range of 10 to 500 cm H₂O. Valve assembly 50 may also be programmed to fully close its valves 47, so as to completely block CSF flow through shunt assembly 30 in order to allow a syringe to penetrate reservoir 36 and extract CSF for analysis or to deliver a therapeutic composition to be infused into reservoir 36. Valve assembly 50 may be positioned anywhere within shunt assembly 30 or reservoir 36, preferably adjacent to shunt outlet 34 or shunt inlet 32.

In an exemplary embodiment shown in FIGS. 3(a) and 3(d), valve assembly 50 may include one or more valves 47 and a separate pump 49. Alternatively, as shown in the exemplary embodiment of FIG. 3(b), one or more valves 47 of valve assembly 50 may be capable of functioning as both a valve and a pump; for example, valve 47 may be configured as an impeller valve. In an exemplary embodiment, valve assembly 50 may be configured as a micropump, preferably a piezoelectric micropump, including a microvalve capable of functioning as a pump or including both a microvalve and/or a micropump. In this embodiment, valve assembly 50 enables fluid to be removed from shunt outlet 34 at a preprogrammed rate to a discharge site, regardless of back pressure. In addition to adjusting the fluid flow rate by opening and closing one or more valves 47 of valve assembly 50, shunt controller 44 may also use pump 49 to force fluid through shunt outlet 34, thereby increasing flow rate. In the same manner that the valves 47 of valve assembly 50 are telemetrically adjusted using reader 52, pump 49 may similarly be remotely programmed to operate at a specific pump rate for a designated period of time. Pump 49 may also be programmed to operate at different pump rates for different designated periods of time. A physician may therefore program controller 44 to run pump 49 at any pump rate for any duration as well as simultaneously or separately adjusting one or more valve of valve assembly 50 for any duration to achieve a desired flow rate and/or fluid pressure. Additionally controller 44 may automatically and continuously adjust pump 49 and the valves 47 of valve assembly 50 to achieve a programmed desired fluid flow rate and/or fluid pressure by comparing the continuously changing fluid pressure and/or flow rate measured by sensor 22 to a preprogrammed desired fluid flow rate and/or fluid pressure and adjusting the pump rate and/or valve opening to achieve the desired fluid flow rate and/or fluid pressure. In an exemplary embodiment, pump 49 may be programmed to remove fluid at a rate of about 20 cc/day. Furthermore, a physician may close the valves 47 of valve assembly 50 and/or stop pump 49 to block CSF flow through shunt assembly 30 to allow a syringe to penetrate the reservoir and extract CSF for analysis or to deliver a therapeutic composition to be infused into reservoir 36.

In addition to valve assembly 50, shunt assembly 30 may further include a supplemental valve 51. As shown in FIG. 3(a), a standard check valve or diaphragm valve 51 may partition reservoir 36 into an inlet chamber 60 and an outlet chamber 62. In an exemplary embodiment, supplemental valve 51 may be configured to open at a specified pressure in the range of 10 to 500 cm H₂O.

As shown in FIG. 1, shunt system 100 further includes a drainage catheter 70 that may be implanted in any bodily region suitable for receiving fluid released from shunt assembly 30. Exemplary drainage sites include, but are not limited to, the peritoneum of the abdomen or the atrium of the heart. Drainage catheter 70 has a tubular body 72 having an outer surface 74, a distal end 76 and proximal end 78. One or more apertures 73 may be positioned anywhere along the length or body of tubular body 72 to allow for egress of spinal fluid. In an exemplary embodiment, drainage catheter 70 may have the same shape, dimension, configuration, and material composition as that of intraspinal catheter 10. Additionally, with the exception of a sensor and conducting wires, drainage catheter 70 may also have the same components as that of intraspinal catheter 10. In an exemplary embodiment, drainage catheter 70 may be about 15 to about 30 cm in length, constructed from a biocompatible non-ferrous radiopaque material and may be more durable and have a high degree of stiffness than intraspinal catheter 10. One or more regions of outer surface 74 along tubular body 72 may be textured to prevent coiling or sliding of tubular body 72 relative to the surrounding tissue. Exemplary textured surfaces include protuberances, such as ribs, nubs, teeth, or barbs, any other roughness means, or combinations thereof. In an exemplary embodiment wherein drainage catheter 70 diverts spinal fluid to the heart, the portion of outer surface 74 positioned within the heart is substantially smooth. For example, about 10 cm or less, preferably, about 7 cm or less of tubular body 72 adjacent to and including proximal end 78 may be substantially smooth.

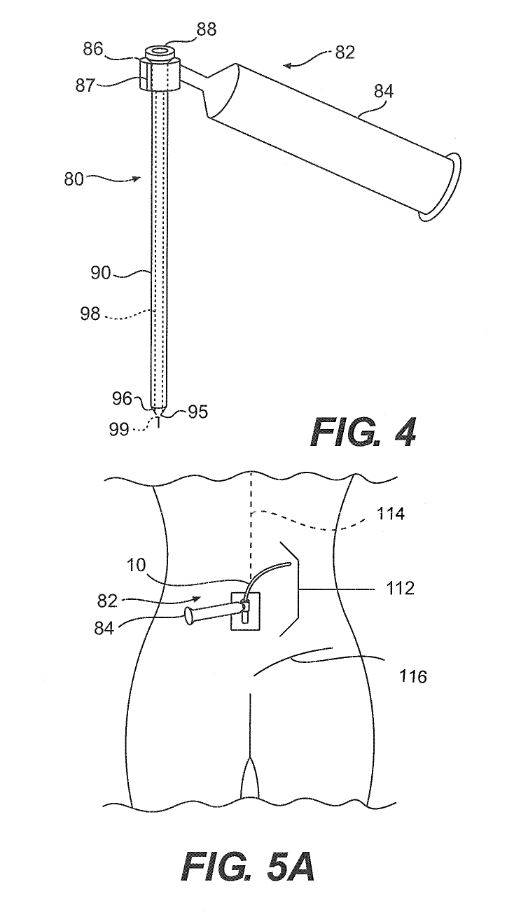

Shunt system 100 of the present invention may be rapidly and safely implanted using a novel introducer assembly 80. As shown in FIG. 4, introducer assembly 80 includes an introducer 82, bore needle 90, and stylet 98. Introducer 82 has an elongated handle 84 that may have any shape, dimension or configuration suitable for facilitating implantation of shunt system 100. A distal end of handle 82 is connected to a sleeve 86 having an introducer channel 88 for receiving one or more medical instruments, such as bore needle 90 and stylet 98. Sleeve 86 may be constructed from any durable material, such as stainless steel. An elongated recess 87 oriented substantially parallel to introducer channel 88 may be positioned within an exterior surface 85 of sleeve 86 to provide a surgical incision guide. Recess 87 may have any shape, preferably a rectilinear shape that is recessed into exterior surface 85 of sleeve 86. In an exemplary embodiment, recess 87 may have a width of about 0.1 mm and a length of about 1.0 cm to about 2.0 cm. In an exemplary embodiment recess 87 substantially extends throughout the length of sleeve 86. Introducer 82 may be configured to have no sharp edges that could inadvertently lacerate the surgeon's gloves or skin or the medical instrument being implanted.

A bore needle 90 may be removably inserted within introducer channel 88 of sleeve 86. Having a substantially straight body 92, bore needle 90 includes a needle channel 93 and conical distal tip 96. Needle channel 93 may be suitably sized to receive various medical instruments, such as stylet 98 and intraspinal catheter 10. A distal tip 96 of bore needle 90 is substantially smooth, having no edges that could cut nerves.

A stylet 98 may be removably inserted within bore needle 90 to reinforce the stiffness of bore needle 90 and to facilitate catheter insertion and placement within an interlaminar space. Stylet 98 may have an elongated body including a distal tip 99 and an electrical potential sensor 95, and as shown in FIG. 4, electrical potential sensor 95 may be embedded in, positioned adjacent to and/or at the tip of distal tip 99. Electrical potential sensor 95 is designed to measure the electrical potential of the environment that surrounding sensor 95, which can indicate the location of distal tip 96 within the body, whether distal tip 96 is in contact with or has damaged a neural structure. Specifically, the electrical potential sensor 95 may assess free run EMG readings or changes in conductance that would indicate contact with a neural element. An exemplary electrical potential sensor 95 may be a simple voltmeter. Electrical potential sensor 95 may be operatively associated with an electrical potential display unit 97 for relaying the electrical potential measurements to a surgeon. Electrical potential sensor 95 may be connected to reader display unit 97 via one or more conduction wires 96 embedded within, positioned along or other wise attached to the body of stylet 98. The wires may run along the substantial length of stylet 98 and extend out from a proximal end of stylet 98, connecting to electrical potential display unit 97.

The present invention is also directed to a novel method for using shunt system 100 and introducer system 80. Shunt system 100 may be used to monitor and regulate ICP, ISP, IVP and/or CSF flow rate that is outside the desired physiological range. Additionally, shunt system 100 may be used to treat conditions wherein abnormal ICP, ISP, IVP or CSF flow rate is an underlying issue, such as abnormally high, abnormally low or abnormally fluctuating ICP, ISP, IVP or CSF flow rate. Additionally, shunt system 100 may be used to treat any disease, syndrome or condition where a higher, lower or otherwise regulated ICP, ISP, IVP or CSF flow rate is desirable. Exemplary conditions for treatment involving an underlying elevated ICP includes benign intracranial hypertension, normal pressure hydrocephalus, tarlov cyst syndrome, chiari malformation, chronic pseudomeningocoele or communicating hydrocephalus. Exemplary conditions for treatment involving an underlying low ICP includes ehlers danlos syndrome. Shunt system 100 may also be use to treat a subset of patients diagnosed with alzheimer's disease, chronic fatigue syndrome, myofascial syndrome, fibromyalgia, or tethered cord syndrome who have an elevated ICP, ISP, IVP or CSF flow rate. For purposes of the present invention, treat or treatment, as used herein refers to any means for producing a beneficial result in an individual affected with an ailment or condition, including but not limited to, effectively reducing the severity of improving, alleviating or curing at least one symptom of an ailment; preventing the onset of an ailment or the manifestation of at least one symptom of an ailment; or combinations thereof.

Shunt system 100 may be implanted in any bodily region of a patient. For purposes of illustration, the following is an exemplary method for using a lumbopaeritoneal shunt embodiment of shunt system 100, wherein intraspinal catheter 10 is implanted in the subarachnoid space 102 of a lumbar vertebra 104 and drainage catheter may be implanted within the peritoneum of the abdomen as shown in FIG. 1.

A patient is first administered local standby or general anesthesia and is positioned on his side, right side up. Preferably, the patient is positioned on a suction bean bag to immobilize the patient. The patient's thighs are flexed at the hip joints to flex and lengthen the lumbar spine. The thorax can also be flexed in instances where shunt system 100 is to be placed into the thoracic spine.

After the patient is prepped and draped, a general surgeon begins anterior to the patient by making a small incision 106 into the abdomen. Drainage catheter 70 is then inserted into incision 106 and laparascopicily placed within the peritoneum of the abdomen.

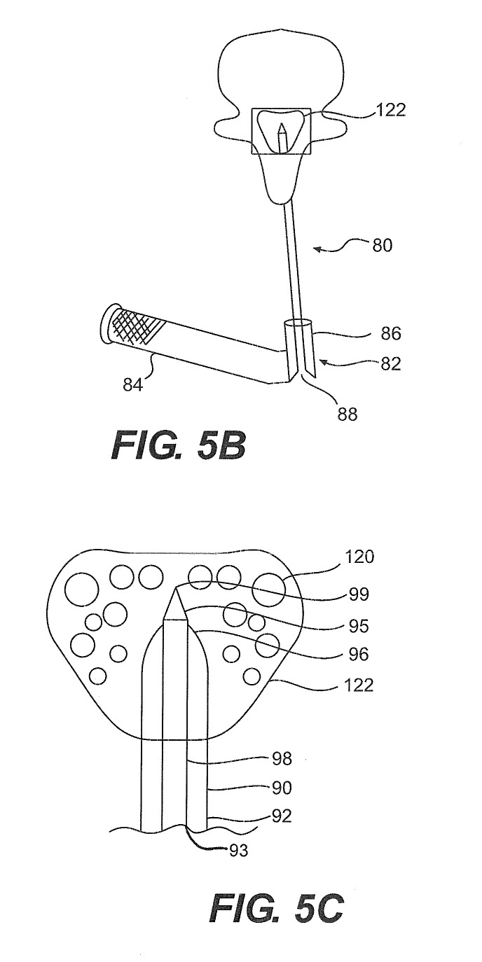

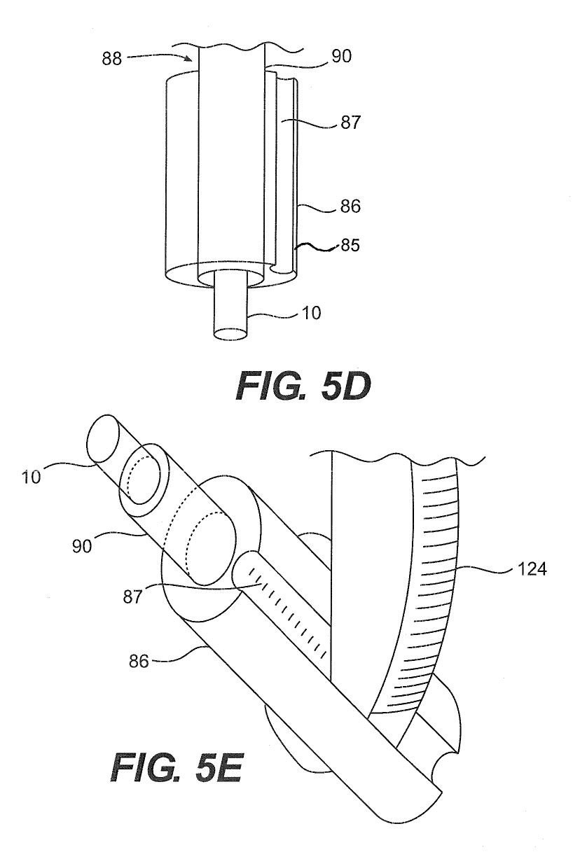

As shown in FIGS. 1 and 5(a), subsequent incisions 108 and 110 are made over the flank of lumbar spine 112 along a midline of the lumbar spinous process 114, above the iliac crest 116. With blunt dissection, a space 118 is made for placement of shunt assembly 30. As shown in FIG. 1, the surgeon then reaches up through space 118 to locate distal end 76 of drainage catheter 70 and places it within the peritoneal cavity using laparoscopic vision. Optionally, a suture may be placed at the level of the peritoneum to stabilize drainage catheter 70.

As shown in FIGS. 5(a)-5(e), intraspinal catheter 10 is subsequently positioned within the lumbar spinal canal using introducer assembly 80. Holding handle 82, a neurosurgeon first inserts bore needle 90 with a centrally positioned stylet 98 into sleeve 86 and drives bore needle 90 and stylet 98 into the lumbar spinal canal from an incision over the midline of the spine. During insertion of bore needle 90, the neurosurgeon may periodically refer to electrical potential display unit 97 to monitor the electrical potential of the tissue surrounding electrical potential sensor 95 positioned at a distal tip 99 of stylet 98. Based on the electrical conductance measurements, the neurosurgeon can determine when distal tip 99 has entered into the subarachnoid space 102 and whether distal tip 99 has contacted any nerves. As shown in FIG. 5(c), a neurosurgeon may rely on the electrical potential reading to safely position bore needle 90 for insertion of intraspinal catheter 10 and avoid potential damage to neural structures, such as nerve roots 120 within dura 122. Bore needle 90 is tilted in the direction of intended intraspinal catheter 10 placement, and stylet 98 is then withdrawn. A manometer may be removably connected to introducer assembly 80 and bore needle 90 to measure spinal fluid pressure. Similarly, a syringe may be removably connected to introducer assembly 80 and bore needle 90 to draw spinal fluid for testing. The stylet is subsequently reintroduced into bore needle 90.

As shown in FIG. 5(e), the neurosurgeon may then use a scalpel 124 to create an incision guided by recess 87 in sleeve 86 of introducer 82. As shown in FIGS. 1 and 5(e), the incision is made transversely from the insertion point of bore needle 90, extending laterally about 2-3 centimeters toward the flank incision. The incision may be used to visualize intraspinal catheter 10 as it is being inserted using introducer assembly 80.

When bore needle 90 has entered the subarachnoid space and is oriented for the proper placement of intraspinal catheter 10, stylet 98 is withdrawn, and intraspinal catheter 10 is inserted through bore needle 90 into the subarachnoid space to a depth of approximately 10 cm to about 15 cm. When properly inserted, CSF should flow out from a distal end 16 of intraspinal catheter 10. Bore needle 90 is then removed, leaving intraspinal catheter 10 in place. Distal end 16 is subsequently tunneled under the skin to the incision in the right flank. Intra-operative fluoroscopy may be used to confirm correct placement of intraspinal catheter 10.

Shunt assembly 30 is then placed within space 118 and deeply seated into the subcutaneous soft tissue within the incisions above the iliac crest. Shunt inlet 32 is friction fitted into or over proximal end 18 of intraspinal catheter 10, and proximal end 78 of drainage catheter 70 subcutaneously tunneled from abdominal laparoscopic incision 106 and similarly friction fitted into or onto shunt outlet 34. The general surgeon then applies gentle traction to intraspinal catheter 10 and drainage catheter 70 to remove any slack from the connected shunt system 100. Fluid drainage into the peritoneal space can be visualized through distal end 76 of drainage catheter 70. The incisions may be subsequently irrigated and closed in two layers, and the wounds may be dressed.

The same implantation methodology can be applied to the thoracic spinal canal. A twist drill may be required to open the inter-laminar space. Additionally, drainage catheter 10 may be placed in the chest cavity, and shunt assembly 30 may be situated over a rib. Furthermore intraspinal catheter 10 is not necessarily limited to placement within the spine. In an exemplary embodiment, intraspinal catheter 10 may be adapted for implant within a ventricle. Similarly, each of the components of shunt system 100 may be adapted to be positioned within any bodily region.

After implantation, shunt assembly 30 can measure intraspinal fluid pressure and/or flow rate and telemetrically transmit the information to reader 52. A surgeon may then use reader 52 to program controller 44 and control dilation of one or more valves 47 of valve assembly 50 and/or pump rate of pump 49. A surgeon may therefore telemetrically program shunt assembly 30 to achieve a desired flow rate and/or fluid pressure for a designated period of time. Additionally, two or more different flow rates and/or fluid pressures may be programmed for different time periods, for example, adjusting for when the patient is reclined as opposed to when the patient is upright. Controller 44 may also be programmed to automatically adjust for a desired flow rate for a predetermined period of time based on real time pressure and/or flow rate measurements obtained from sensor 22.

Shunt system 100 may also be used as a drug delivery means. In an exemplary embodiment, the radiopaque reservoir, radiopaque shunt inlet 32 and radiopaque shunt outlet 34 of shunt assembly 30 provide a viewable target for the surgeon under fluoroscopy. As shown in FIG. 3(a), the surgeon may aim for target 126. A needle may be inserted through intraspinal catheter 10 or drainage catheter 70 and inject a solid or fluid into reservoir 36. Exemplary therapeutic materials that may be injected into shunt assembly 30 include drugs, nerve growth factor, and antibiotics. Therefore in addition to regulating ICP, ISP, IVP and CSF flow rate, shunt system 100 may be an effective therapeutic delivery system useful in treating a wide range of diseases, including but not limited to carcinomatous meningitis, multiple sclerosis, various types of cancers and hormone deficiencies.

Shunt system 100 of the present invention is unique in that it is a fully modular, MRI compatible/visible, telemetric system capable of measuring, automatically regulating and extracorporeally adjusting CSF flow and/and pressure. The modular property of shunt system 100 allows for rapid and safe replacement of intraspinal catheter 10, shunt assembly 30 and/or drainage catheter 70. Shunt system 100 also need not be implanted or placed according to a specific orientation, thereby further simplifying the implantation and replacement process. Additionally, one or more radiopaque portions of, preferably, all the components of, shunt system 100 enables visualization of the shunt system 100 and components thereof under MRI to confirm that shunt system 100 is properly positioned as well as to facilitate procedures that may be performed under intraoperative fluoroscopy, such as manometry or drawing spinal fluid from shunt assembly 30.

Shunt system 100 is also telemetric, allowing for extracorporeal monitoring and adjustment of fluid flow. Specifically, induction coil transponder 46 may be used to continuously or periodically monitor the CSF flow rate, ICP, ISP and/or IVP measured by sensor 22. By remotely controlling valve assembly 50, including the valves 47 and/or pump 49 therein, it is possible to increase drainage of spinal fluid to lower intraspinal and intracranial cerebrospinal fluid pressures, substantially block drainage, thereby preventing over drainage, or otherwise regulate fluid flow. Additionally, fluid may be infused into shunt assembly 30 by introducing a needle through a catheter and shunt inlet 32 or shunt outlet 34 to treat chronic conditions such as cancer, infection, and degenerative disease.

A number of other features of shunt system 100 also simplify and increase the safety and efficacy of surgical placement of shunt system 100. For example, approximately 5% of lumbar shunt insertions contact or penetrate the spinal cord or nerves. Using the novel introducer assembly 80 of the present invention, contact with neural structures may be avoided by monitoring the electrical resistance of the tissue adjacent to electrical potential sensor 95 positioned on a distal tip of stylet 98. Alternatively, sensor 95 can produce a voltage of about 20 milli-amps or less to stimulate the surrounding tissue and measure the resulting electrical resistance to determine the type of tissue adjacent to sensor 95, specifically whether it is neural tissue.

Additionally, shunt system 100 is configured as a simple device having a minimal number of components that may be rapidly and securely attached to one another and to an implant site. For example, fastening structures 35 of shunt inlet 32 and shunt outlet 34 and/or corresponding mating structure of intraspinal catheter 10 and drainage catheter 70 enhance attachment between the components of shunt system 100. Additionally, the textured surface area of intraspinal catheter 10, drainage catheter 70 and shunt assembly 30 prevents catheter coiling of and anchors shunt system 100 relative to the adjacent bodily tissue, obviating or minimizing the need for suturing.

These and various other advantages and features of novelty that characterize the invention are pointed out with particularity in the claims annexed hereto and forming a part hereof. However, for a better understanding of the invention, its advantages, and the objects obtained by its use, reference should be made to the drawings which form a further part hereof, and to the accompanying descriptive matter, in which there is illustrated and described a preferred embodiment of the invention.

It is to be understood that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

What is claimed is:

1. A shunt system comprising:
a first catheter comprising:
a first tube body; and
a sensor positioned on the first tube body; and
a shunt assembly configured to be surgically implanted in a bodily cavity and removably attached to the first catheter, wherein the shunt assembly comprises:
a reservoir;
a valve assembly that controls fluid flow through the shunt assembly; and
a first controller operatively associated with the valve assembly and sensor; and
a second catheter removably attached to the shunt assembly comprising a second tube body.

2. The shunt system of claim 1, wherein an exterior surface of the first tube body or second tube body has a textured region selected from the group consisting of: protuberances, ribs, nubs, teeth, barbs, and combinations thereof.

3. The shunt system of claim 1, further comprising a wire embedded within, attached to or positioned along the first tube body connecting the valve assembly and controller.

4. The shunt system of claim 1, wherein a portion of the first tube body comprises a tubular elbow bent at a prefabricated angle.

5. The shunt system of claim 4, wherein said angle is about 45 degrees to about 120 degrees.

6. The shunt system of claim 1, wherein the shunt assembly further comprises a shunt inlet and a shunt outlet, wherein a surface of the shunt inlet or shunt outlet comprises a plurality of protuberances suitable to facilitate attachment to the first proximal end or the second proximal end.

7. The shunt system of claim 1, wherein said shunt assembly further comprising a supplemental valve that partitions the reservoir into a shunt inlet chamber and shunt outlet chamber.

8. The shunt system of claim 1, wherein the valve assembly comprises a pump capable of driving fluid through the shunt outlet.

9. The shunt system of claim 8, wherein the valve assembly is configured as a piezoelectric micropump.

10. The shunt system of claim 1, wherein said shunt assembly further comprises:
a first signal conditioner; and
a first transponder operatively associated with the first signal conditioner and sensor,
wherein the first transponder is capable of transmitting a signal encoding pressure or flow rate measurements obtained from the sensor; and
wherein the shunt system further comprises a reader assembly positioned outside of the bodily cavity, wherein the reader assembly comprises:
a second signal conditioner;
a remote second transponder operatively associated with the second signal conditioner and
capable of receiving the transmitted signal encoding pressure or flow rate measurements obtained from the sensor; and
a second controller operatively associated with the second transponder.

11. The shunt system of claim 10, wherein the first transponder and the second transponders are each configured as a magnetic induction coil.

12. The shunt system of claim 11, wherein the shunt assembly further comprises a rechargeable battery and wherein the battery may be remotely charged using the magnetic induction coil of the reader assembly.

13. The shunt system of claim 1, wherein the first catheter, shunt assembly or second catheter is constructed from a radiopaque and biocompatible material.

14. A shunt system comprising:
a shunt assembly configured to be surgically implanted in a bodily cavity, wherein the shunt assembly comprises:
a valve assembly that controls fluid flow through the shunt assembly, wherein said
valve assembly comprises a pump capable of forcing fluid out of the shunt assembly;
a first controller operatively associated with the valve assembly;
a sensor capable of measuring pressure or flow rate, wherein the sensor is operatively associated with the controller;
a first signal conditioner operatively associated with the controller; and
a first transponder operatively associated with the first signal conditioner and capable of receiving a signal; and
a reader assembly positioned outside the bodily cavity, wherein the reader assembly comprises:
a second signal conditioner;
a second transponder unit operatively associated with the second signal conditioner and capable of transmitting the signal; and
a second controller operatively associated with the second signal conditioner.

15. The shunt system of claim 14, wherein the first transponder and the second transponders are each configured as a magnetic induction coil.

16. The shunt system of claim 14, wherein the shunt assembly further comprises a rechargeable battery and wherein the battery may be remotely charged using the magnetic induction coils of the reader assembly.

17. The shunt system of claim 14, wherein the valve assembly is a piezoelectric micropump.

18. The shunt system of claim 14, further comprising a supplemental valve that partitions the reservoir into a shunt inlet chamber and shunt outlet chamber.

19. An introducer system comprising:
an introducer adapted to introduce a medical device into a body, wherein the introducer comprises:
a handle;
a sleeve attached to a distal end of the handle, wherein the sleeve comprises:
an introducer channel for receiving the medical instrument; and
a recess positioned on an exterior surface of the sleeve, wherein the recess is substantially parallel to the introducer channel and configured to guide surgical incisions.

20. The introducer system of claim 19, further comprising
a needle removably positioned within the introducer channel, wherein the needle comprises a needle channel; and
a stylet removably received within the needle channel, wherein the stylet comprises a sensor for detecting an electrical potential of a tissue surrounding the sensor.
